(19) United States
(12) Patent Application Publication
(10) Pub. No.: US 2023/0309925 A1
(43) Pub. Date: Oct. 5, 2023

(54) BIOLOGICAL-INFORMATION ACQUISITION DEVICE, BIOMETRIC SENSOR UNIT, AND MAIN BODY UNIT

(71) Applicant: Murata Manufacturing Co., Ltd., Nagaokakyo-shi (JP)

(72) Inventors: Masahiro SASAKI, Nagaokakyo-shi (JP); Masashi FURUKUBO, Nagaokakyo-shi (JP)

(21) Appl. No.: 18/327,309

(22) Filed: Jun. 1, 2023

Related U.S. Application Data
(63) Continuation of application No. PCT/JP2021/039249, filed on Oct. 25, 2021.

(30) Foreign Application Priority Data
Dec. 24, 2020 (JP) 2020-214673

(51) Int. Cl. A61B 5/00

(52) U.S. Cl. CPC A61B 5/6802; A61B 5/002; A61B 5/6826; A61B 2560/0443; A61B 2560/0214; A61B 2560/0456; A61B 2562/08

(57) ABSTRACT

A biological-information acquisition device is provided that includes a biometric sensor part including a biometric sensor that detects biological information and a living-body wearable part that enables the biometric sensor part to be in contact with a living body and a main body part including an information processing section that processes biological information and a battery that supplies power. The biometric sensor part is configured to be replaceable by being separated from the main body part, and the main body part is detachably attached to the biometric sensor in such a manner as not to come into contact with a living body when the biometric sensor part is worn on the living body.

### CROSS REFERENCE TO RELATED APPLICATIONS

This application is a continuation of PCT Application No. PCT/JP2021/039249, filed Oct. 25, 2021, which claims priority to Japanese Patent Application No. 2020-214673, filed Dec. 24, 2020, the entire contents of each of which are hereby incorporated by reference in their entirety.

### TECHNICAL FIELD

The present invention relates to a biological-information acquisition device, a biometric sensor unit, and a main body unit. The biometric sensor unit and the main body unit are configured to be used in the biological-information acquisition device.

### BACKGROUND

In recent years, various biometric sensors that acquire biological information such as pulse and electrocardiogram by being worn on a living body have been proposed. For example, Japanese Patent Application Publication No. 2001-70264 (hereinafter “Patent Document 1” discloses a ring-type pulse wave sensor including a sensor main body with a built-in circuit unit, a detection unit that detects pulse-wave information of a human body, and a belt or a clip that fixes the sensor main body onto a finger, the sensor main body and the detection unit being flexibly connected to each other by a printed circuit board having flexibility.

Moreover, Japanese Patent Application Publication No. 11-332840 (hereinafter “Patent Document 2”) discloses a ring-type photoplethysmographic signal detector including a wearable portion that has a partially cut annular shape and that serves as a finger ring and an adjustment mechanism (e.g., a constant-load spring) that entirely reduces the diameter of the inner peripheral surface of the wearable portion.

In a device that acquires biological information by using a biometric sensor, a sensor portion that comes into contact with a living body (e.g., a human body) is likely to be contaminated with, for example, sweat or oil on the skin, and thus, there is a problem in that the service life of the device may be shortened. In addition, there may be a case where measurement becomes unstable or cannot be performed depending on the situation in which the device is used, individual differences, such as individual characteristics, and so forth. More specifically, for example, in the ring-type biometric sensors (e.g., biometric sensors to be worn on a finger) disclosed in Patent Documents 1 and 2 or the like, there may be a case where measurement becomes unstable or cannot be performed due to, for example, finger size, skin color, the influence of body movement, or the like. On the other hand, if such biometric sensors are replaced (e.g., selectively used) in accordance with various usage situations or individual differences, the cost will increase.

### SUMMARY OF THE INVENTION

Accordingly, it is an object of the present invention to provide a biological-information acquisition device that prevents contamination of a main body part and flexibly adapting to various usage situations and individual differences while suppressing an increase in the manufacturing costs, a biometric sensor unit used in the biological-information acquisition device, and a main body unit.

In an exemplary aspect, a biological-information acquisition device is provided that includes a biometric sensor unit including a biometric sensor that detects biological information and a living-body wearable part that enables the biometric sensor to be in contact with a living body and a main body including an information processor that processes the biological information and a battery that supplies power to the information processor. The biometric sensor unit is configured to be replaceable by being separated from the main body. The main body is detachably attached to the biometric sensor unit in such a manner as not to come into contact with a living body when the biometric sensor unit is worn on the living body.

According to the exemplary aspect of the biological-information acquisition device, the biometric sensor unit, which is wearable on a living body, is configured to be replaceable by being separated from the main body part. In other words, only the biometric sensor unit can be replaced, and the main body can be reused. This configurations enables easy replacement of the biometric sensor unit and changing of the wearing form of the biometric sensor unit while reducing the manufacturing costs. In addition, the main part is detachably attached to the biometric sensor unit in such a manner as not to come into contact with a living body when the biometric sensor unit is attached to the living body. Therefore, contamination of the main body can be prevented.

As a result of the exemplary configuration according to the present invention, contamination of the main body can be prevented, and the biological-information acquisition device can be flexibly adapted to various usage situations and individual differences while suppressing an increase in the manufacturing costs.

### BRIEF DESCRIPTION OF DRAWINGS

FIG. 1 is an external view illustrating an appearance of a biological-information acquisition device according to an exemplary embodiment.

FIG. 2 is a sectional view illustrating the biological-information acquisition device according to the exemplary embodiment.

FIG. 3 is a perspective view illustrating (a diagram illustrating in a see-through manner) the biological-information acquisition device according to the exemplary embodiment.

FIG. 4 is an external view (a perspective view) illustrating an appearance of a biometric sensor part that is included in the biological-information acquisition device according to the exemplary embodiment.

FIG. 5 is an external view (a perspective view) illustrating an appearance of the biometric sensor part included in the biological-information acquisition device according to the exemplary embodiment.

FIG. 6 is an external view (a plan view) illustrating a main body part that is included in the biological-information acquisition device according to the exemplary embodiment.

FIG. 7 is an external view (a perspective view) illustrating the main body part included in the biological-information acquisition device according to the exemplary embodiment.

FIG. 8 is a perspective view illustrating the interior (a top surface side) of the main body part included in the biological-information acquisition device according to the exemplary embodiment.

FIG. 9 is a perspective view illustrating the interior (a bottom surface side) of the main body part included in the biological-information acquisition device according to the exemplary embodiment.

FIGS. 10(a) and 10(b) are diagrams illustrating the bottom surface (external terminals) of the main body part included in the biological-information acquisition device according to the exemplary embodiment. In particular, FIG. 10(a) illustrates the main body part in the process of being set into a charger, and FIG. 10(b) illustrates the main body part set in the charger.

FIG. 11 is a diagram illustrating an example of how to wear the ring-type biological-information acquisition device according to the exemplary embodiment.

FIG. 12 is a diagram illustrating an example of how to wear an earphone-type biological-information acquisition device according to a modification of the exemplary embodiment.

FIG. 13 is a perspective view illustrating an appearance of the charger that charges the main body part (a battery) included in the biological-information acquisition device according to the exemplary embodiment.

FIG. 14 is a diagram illustrating a state in which the main body part included in the biological-information acquisition device according to the exemplary embodiment is in the process of being set into the charger.

FIG. 15(a) is a side view illustrating the main body part, which is included in the biological-information acquisition device according to the exemplary embodiment, in the process of being set into the charger, and FIG. 15(b) is a view illustrating in a see-through manner the main body part set in the charger.

### DETAILED DESCRIPTION OF EMBODIMENTS

An exemplary embodiment of the present invention will be described in detail below with reference to the drawings. Note that, in the drawings, portions that are the same as each other or that correspond to each other are denoted by the same reference sign. In addition, in the drawings, the same elements are denoted by the same reference signs, and repeated descriptions will be omitted.

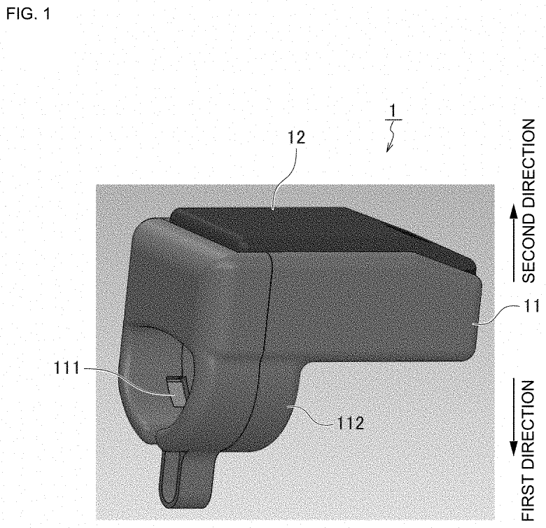

The configuration of a biological-information acquisition device (device) 1 according to the exemplary embodiment will be described first with reference to FIG. 1 to FIG. 15.

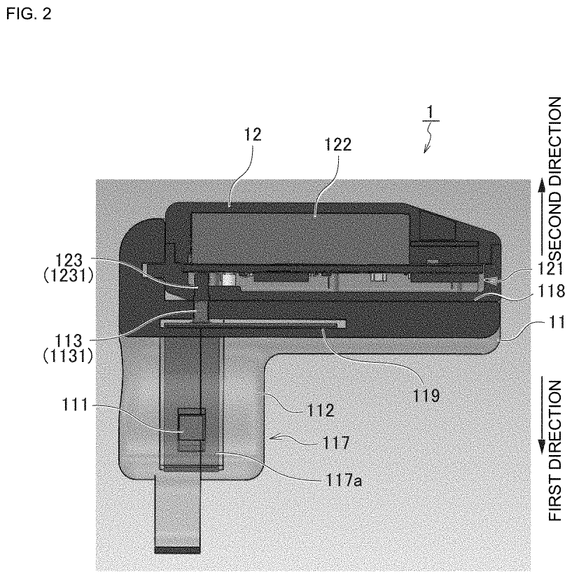

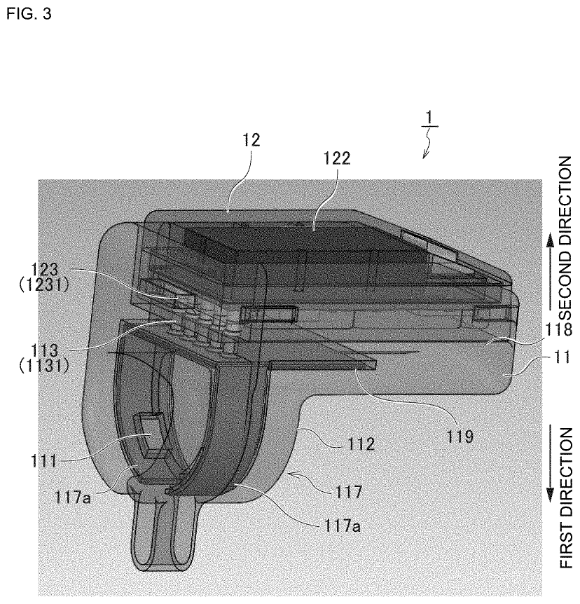

According to the exemplary aspect, the biological-information acquisition device 1 is configured to acquire and process biological information and wirelessly output (e.g., transmits) a result of processing the biological information. As illustrated in FIG. 1 to FIG. 3, the biological-information acquisition device 1 includes a biometric sensor part (biometric sensor unit) 11 and a main body part (main body unit) 12. Note that FIG. 1 is an external view illustrating an appearance of the biological-information acquisition device 1. FIG. 2 is a sectional view illustrating the biological-information acquisition device 1. FIG. 3 is a perspective view illustrating (a diagram illustrating in a see-through manner) the biological-information acquisition device.

In particular, the biological-information acquisition device 1 is configured to prevent contamination of the main body part (also referred to as a main body unit or simply a main body) 12 and has an ability to flexibly adapt to various usage situations and individual differences while suppressing an increase in the manufacturing costs. Thus, the biometric sensor part (also referred to as a biometric sensor unit) 11 is configured to be replaceable by being separated from the main body part (main body unit) 12.

It is noted that the biometric sensor part 11 can be solely distributed, and in this case, it is referred to as the biometric sensor unit 11 for differentiation. Accordingly, the biometric sensor part 11 and the biometric sensor unit 11 are the same as each other. Similarly, the main body part 12 (i.e., the main body) can be solely distributed, and in this case, it is referred to as the main body unit 12 for differentiation. Accordingly, the main body part 12 and the main body unit 12 are the same as each other.

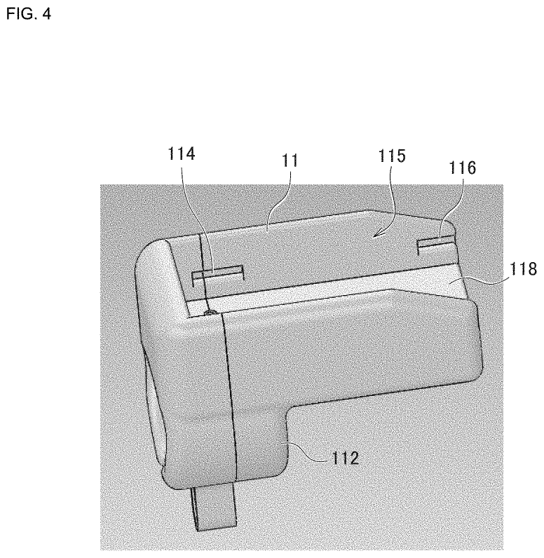

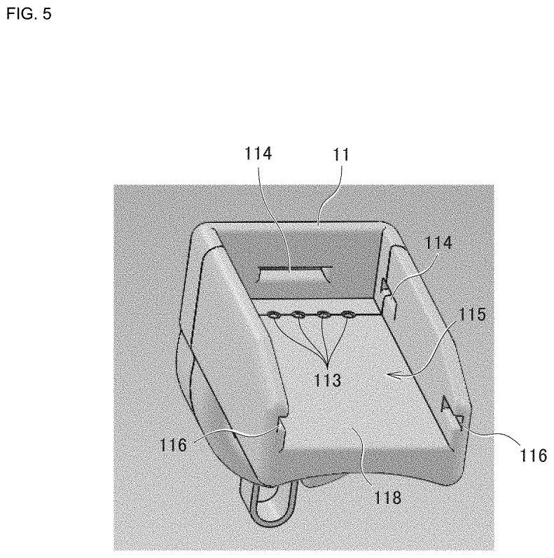

As illustrated in FIG. 1 to FIG. 5, the biometric sensor part 11 mainly includes biometric sensors 111 that are configured to detect biological information and a living-body wearable part 112 that enables and configures the biometric sensors 111 to be in contact with a living body. Note that FIG. 4 and FIG. 5 are each an external view (e.g., perspective views) illustrating an appearance of the biometric sensor part 11.

According to exemplary aspects, examples of the biological information include body temperature, deep body temperature, blood pressure, blood glucose level, oxygen saturation, heart rate, pulse rate, ECG, and PPG. In other words, various biometric sensors (e.g., a body temperature sensor, a photoplethysmographic sensor, a pressure sensor, a blood glucose level sensor, an oxygen saturation sensor, a pulse wave sensor, and an electrocardiographic electrode) that are configured to detect (e.g., acquire) these biological information items are used as the biometric sensors 111.

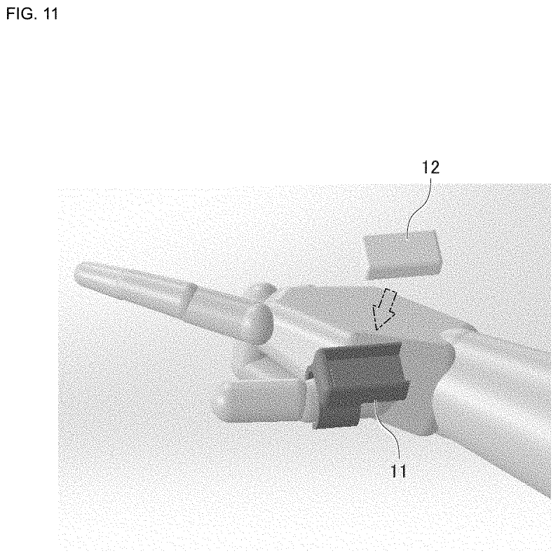

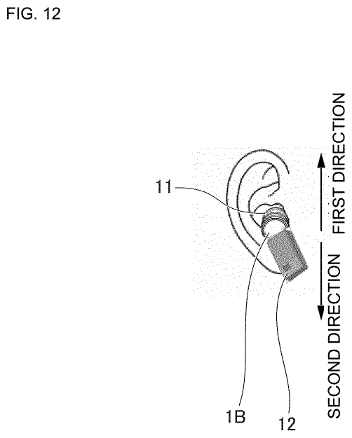

According to various exemplary aspects, the living-body wearable part 112 can be, for example, a ring-shaped (a ring-type) living-body wearable part 112 that is to be worn on a finger (see FIG. 11), an earphone-shaped (an earphone-type) living-body wearable part 112 that is to be worn on an ear (see FIG. 12), or an attachment-type (a patch-type) living-body wearable part 112 that is attachable to skin. In addition, it is preferable that there be a variety of living-body wearable parts 112 having different shapes and sizes in order to, for example, flexibly adapt to usage situations, individual differences (individual physical characteristics), and so forth. In the present embodiment, the ring-type biometric sensor part 11 will be described as an example.

Note that, in the case of the earphone-type biometric sensor part 11 that is to be worn on an ear, as illustrated in FIG. 12, a portion of the biometric sensor part 11 protrudes and is exposed in a first direction (i.e., a direction in which a living body wearing the biometric sensor part 11 is located when viewed from the main body part 12). The main body part 12 is not exposed in the first direction and is exposed in a second direction that is opposite to the first direction. With this configuration, contamination of the main body part 12 is prevented. In addition, by employing the configuration in which the main body part 12 is not exposed in the first direction but exposed in the second direction, even when the main body part 12 is replaced in a state where the biological-information acquisition device 1 is worn by a living body, the replacement can be easily performed while preventing contamination of the main body part 12.

In the case of the attachment-type (e.g., patch-type) biometric sensor part 11 that is attachable to skin, it has an attachment surface attachable to skin, and the attachment surface is exposed in the first direction that is perpendicular to the attachment surface. The main body part 12 is positioned so as not to be exposed in the first direction but to be exposed in the second direction, which is opposite to the first direction. With this configuration, contamination of the main body part 12 is prevented. In addition, by employing the configuration in which the main body part 12 is not exposed in the first direction but exposed in the second direction, even when the main body part 12 is replaced in a state where the biological-information acquisition device 1 is worn by a living body, the replacement can be easily performed while preventing contamination of the main body part 12.

Note that it has been described that the main body part 12 is not exposed in the first direction both in the case of the earphone-type biometric sensor part 11 and the case of the patch-type biometric sensor part 11, and the main body part 12 is not exposed even in the case where a small through hole or cutout portion that corresponds to a button (e.g., a reset button or the like) of the main body part 12 is formed in the biometric sensor part 11 so as to extend in the first direction. This is because, if such a through hole or cutout portion is small, contaminants stop at an inner wall and will not reach the main body part 12, and thus, advantageous effects of the present invention can be obtained.

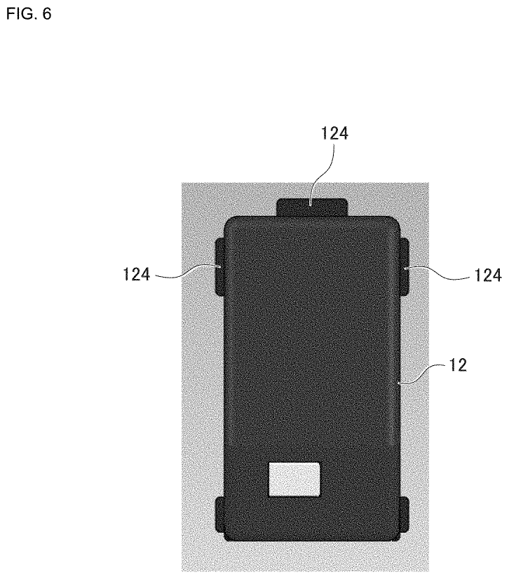

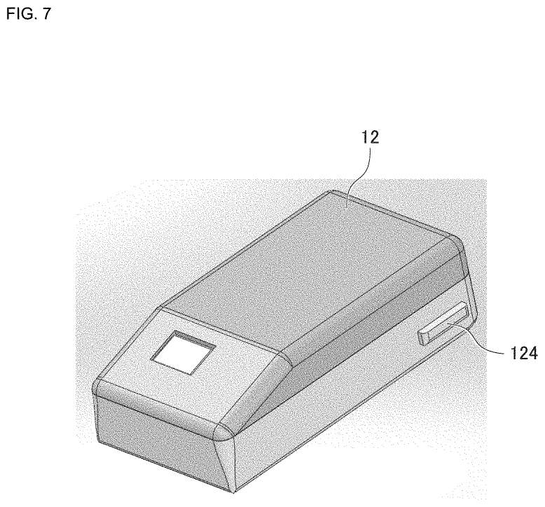

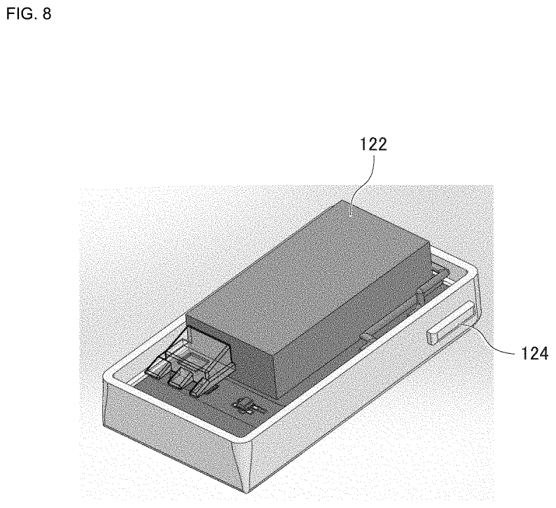

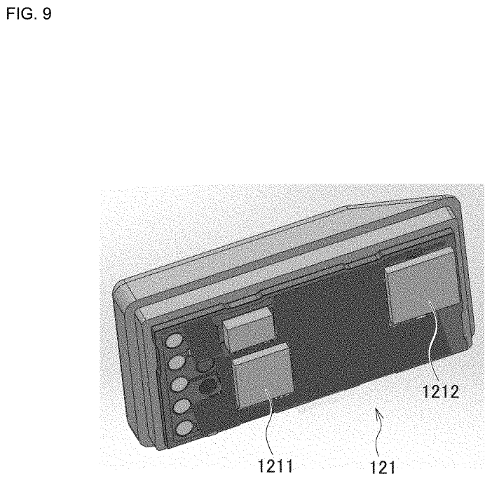

According to an exemplary aspect, the main body part 12 is detachably attached to the biometric sensor part 11 so as not to come into contact with a living body (e.g., a finger) when the biometric sensor part 11 is attached to the living body (see FIG. 11 and FIG. 12). As illustrated in FIG. 1 to FIG. 3 and FIG. 6 to FIG. 9, the main body part 12 mainly includes an information processing section (e.g., information processing circuit or processor) 121 that is configured to process the biological information acquired by the biometric sensor part 11 and output (e.g., transmit) a result of processing the biological information and a battery 122 that supplies power to the information processing section 121 and supplies power (as necessary) to the biometric sensors 111 of the biometric sensor part 11 and so forth. Note that FIG. 6 is an external view (e.g., a plan view) illustrating the main body part 12. FIG. 7 is an external view (e.g., a perspective view) illustrating the main body part 12. FIG. 8 is a perspective view illustrating the interior (e.g., a top surface side) of the main body part 12. FIG. 9 is a perspective view illustrating the interior (e.g., a bottom surface side) of the main body part 12.

Regardless of the type of the biometric sensor part 11, that is, regardless of whether the biometric sensor part 11 is a ring-type, an earphone-type, or an attachment-type biometric sensor part 11, the same main body part 12 can be used. In other words, the main body part 12 has a structure that is connectable to different types of biometric sensor parts 11, such as the ring-type biometric sensor part 11 or the earphone-type biometric sensor part 11. Thus, biological information (e.g., vital data) can be acquired from various parts of a living body by attaching the main body part 12 to the different biometric sensor parts 11. In addition, the measuring position can be changed in accordance with the environment.

In an exemplary aspect, the information processing section 121 of the main body part 12 includes, in addition to a micro control unit (MCU) that executes information processing, a memory (corresponding to a storage unit described in the claims) 1211 such as an EEPROM that stores identification information (ID) of a user (a patient), identification information (ID) of the biological-information acquisition device 1, a program for causing the MCU to execute processing of biological information, and so forth, and a wireless communication module 1212 (corresponding to a wireless communication unit described in the claims) such as a BLE chip that transmits (outputs) a processing result of biological information. It is also noted that, by storing identification information in the main body part 12, even if the biometric sensor part 11 is replaced, the biological-information acquisition device 1 can be used in the same manner without changing the personal recognition or the like.

According to an exemplary aspect, wireless communication uses a wireless communication system (wireless communication standard) such as the Bluetooth® low energy (BLE), WiFi, long term evolution (LTE), or sub-GHz (bandwidth of 900 MHz). Alternatively, for example, a wireless communication system (wireless communication standard), such as near field communication (NFC) (ISO/IEC 18092) or MIFARE® (ISO/IEC 14443) may be used.

The biometric sensor part 11 includes a plurality of sensor-side external terminals (e.g., connection terminals) 113 that are electrically connected to the main body part 12. Similarly, the main body part 12 includes a plurality of main-body-side external terminals (e.g., connection terminals) 123 that are electrically connected to the biometric sensor part 11. In other words, the biometric sensor part 11 and the main body part 12 are electrically connected to each other by the plurality of sensor-side external terminals 113 and the plurality of main-body-side external terminals 123 in the exemplary embodiment.

Preferably, the plurality of sensor-side external terminals 113 and the plurality of main-body-side external terminals 123 are arranged in or on surfaces that face each other in a direction perpendicular to directions in which the main body part 12 is attached and detached to and from (is inserted and extracted into and from) the biometric sensor part 11 in order to reduce the probability of occurrence of a contact failure.

More specifically, the plurality of sensor-side external terminals 113 are arranged in a row in or on the top surface (e.g., the upper surface) of a flat portion 118 of the biometric sensor part 11. In contrast, the plurality of main-body-side external terminals 123 are arranged in a row in or on the bottom surface of the main body part 12 (see FIG. 5 and the like). Here, the battery 122 of the main body part 12 is disposed above the main-body-side external terminals 123 (see FIG. 2 and the like). With this configuration, the battery 122 which is relatively heavy is located above the main-body-side external terminals 123, and thus, connection between terminal portions becomes mechanically and electrically stable.

In addition, each of the sensor-side external terminals 113 are preferably provided with biasing members 1131 (e.g., coil springs or plate springs) that bias the sensor-side external terminals 113 in a direction in which the sensor-side external terminals 113 come close to the main-body-side external terminals 123 (e.g., an outward direction). Similarly, it is preferable that the main-body-side external terminals 123 be provided with biasing members 1231 (e.g., coil springs or plate springs) that bias the main-body-side external terminals 123 in a direction in which the main-body-side external terminals 123 come close to the sensor-side external terminals 113 (e.g., an outward direction). It is noted that only the sensor-side external terminals 113 may be provided with the biasing members 1131, or only the main-body-side external terminals 123 may be provided with the biasing members 1231.

If the rigidity of each of the sensor-side external terminals 113 is high while the biometric sensor part 11 has elasticity, and a stress is applied, there is a possibility that the stress may be concentrated at the sensor-side external terminals 113. However, by providing the biasing members 1131/1231, the probability of such stress concentration can be reduced.

In an exemplary aspect, the main body part 12 is engaged with (e.g., fitted into) the biometric sensor part 11 at, for example, three points and secured to the biometric sensor part 11. More specifically, in an exemplary aspect, the main body part 12 is formed in a substantially cubic shape and includes three hook portions 124 that are formed in a protruding manner on an end outer surface and left and right outer surfaces (e.g., outer sides) thereof (see FIG. 6 and the like). Thus, when the main body part 12 is attached or detached, it can be reliably fixed in an accurate position or can be easily detached.

In contrast, a housing of the biometric sensor part 11 is made of a material having elasticity and has three recesses 114 into which the hook portions 124 of the main body part 12 are fitted. More specifically, the biometric sensor part 11 has a recessed accommodating portion 115 in which the main body part 12 is to be accommodated, and the above-mentioned three recesses 114 are formed on an inner surface of the accommodating portion 115 (see FIG. 5 and the like). Thus, attachment and detachment (removal and fitting) of the main body part 12 are easily performed, and the main body part 12 is less likely to break even if attachment and detachment of the main body part 12 are repeatedly performed.

In addition to the above-mentioned three recesses 114, guide grooves 116 for guiding the hook portions 124 of the main body part 12 to the accommodating portion 115 are formed in end portions of the inner surface of the accommodating portion 115. In this exemplary aspect, the guide grooves 116 preferably are formed so as to be inclined with respect to the bottom surface of the accommodating portion 115 (so as to be sloped). With this configuration, the biometric sensor part 11 and the main body part 12 can be positioned with respect to each other by the guide grooves 116 and easily connected to each other. In addition, the main body part 12 can be fixed in a suitable position.

The living-body wearable part 112 of the biometric sensor part 11 includes a curved portion 117 formed to be curved so that it is wearable on a finger of a living body and the flat portion (e.g., a planar portion) 118 formed so as to connect one end and the other end of the curved portion 117 to each other. The curved portion 117 is provided with a flexible substrate 117a that has flexibility and that is formed in a belt-like shape and the two biometric sensors 111 that are attached to portions close to the ends of the flexible substrate 117a (see FIG. 3 and the like). The biometric sensors 111 are electrically connected to the main body part 12 by the flexible substrate 117a, the sensor-side external terminals 113, and the main-body-side external terminals 123, and an electrical signal (e.g., a voltage or the like) that corresponds to biological information is read by the main body part 12.

As illustrated in FIGS. 1, 2, and 3, the curved portion 117, which is a portion of the biometric sensor part 11, protrudes and is exposed in the first direction perpendicular to the flat surface of the flat portion 118 (the direction in which a living body wearing the biometric sensor part 11 is located when viewed from the main body part 12). Moreover, the main body part 12 is not exposed in the first direction, but it is exposed in the second direction, which is opposite to the first direction. With this configuration, contamination of the main body part 12 can be prevented. In addition, by employing the configuration in which the main body part 12 is not exposed in the first direction, but is exposed in the second direction, even when the main body part 12 is replaced in a state where the biological-information acquisition device 1 is worn by a living body, the replacement can be easily performed while preventing contamination of the main body part 12.

It has been described above that the main body part 12 is not exposed in the first direction, and the main body part 12 is not exposed even when a small through hole or cutout portion that corresponds to a button (e.g., a reset button or the like) of the main body part 12 is formed in the biometric sensor part 11 so as to extend in the first direction. This is because, if such a through hole or cutout portion is small, contaminants stop at an inner wall and will not reach the main body part 12, and thus, advantageous effects of the present invention can be obtained.

By providing the flat portion 118, when the biometric sensor part 11 is worn on a finger, the biometric sensor part 11 can be prevented from rotating or being incorrectly worn, and the biometric sensors 111 can be maintained at suitable positions. In addition, by arranging the biometric sensors 111 at the curved portion 117 that is easily brought into close contact with a living body, accurate measurement can be performed. Furthermore, by arranging the flexible substrate 117a at the curved portion 117, the biometric sensors 111 can be brought into close contact with a finger by the elasticity of the flexible substrate 117a.

Moreover, in the exemplary aspect, the curved portion 117 is made of an elastic material, and, therefore, has elasticity. In contrast, the flat portion 118 has a rigidity higher than that of the curved portion 117. By forming the curved portion 117 by using an elastic material, for example, it can accommodate different finger sizes. In addition, by increasing the rigidity of the flat portion 118 so as to maintain the shape of the flat portion 118, the sensor positions can be maintained.

Moreover, it is preferable that the flat portion 118 further include a reinforcing plate 119 (see FIG. 2, FIG. 3, and the like). By including the reinforcing plate 119 in the flat portion 118, the shape of the flat portion 118 can be maintained, and breakage of the flat portion 118 due to stress or the like can be prevented.

It is also preferable that the flat portion 118 have a dimension (e.g., a width) larger than that of the curved portion 117 in the axial direction of the curved portion 117 (the axial direction of a finger on which the living-body wearable part 112 is worn). With this configuration, the stress applied to the sensor-side external terminals 113 is received by the flat portion 118 in a dispersed manner, so that the rigidity can be ensured. In addition, for example, there are the following advantages: the flat portion 118 can be easily held, the flat portion 118 can be easily bent at an interface between the flat portion 118 and the curved portion 117, and the flat portion 118 can be easily recognized as a mark.

Moreover, in the exemplary aspect, the battery 122 included in the main body part 12 is formed in, for example, a rectangular parallelepiped shape and is preferably disposed in such a manner that the longitudinal direction thereof is parallel to the axial direction of the curved portion 117 of the biometric sensor part 11 (the axial direction of a finger on which the living-body wearable part 112 is worn).

The main body part 12 further includes charging external terminals (e.g., connection terminals) 125 that are electrically connected to (i.e., connectable to) a charger 100 that charges the battery 122. Thus, only the main body part 12 that does not come into contact with a living body can be detached and charged hygienically and easily.

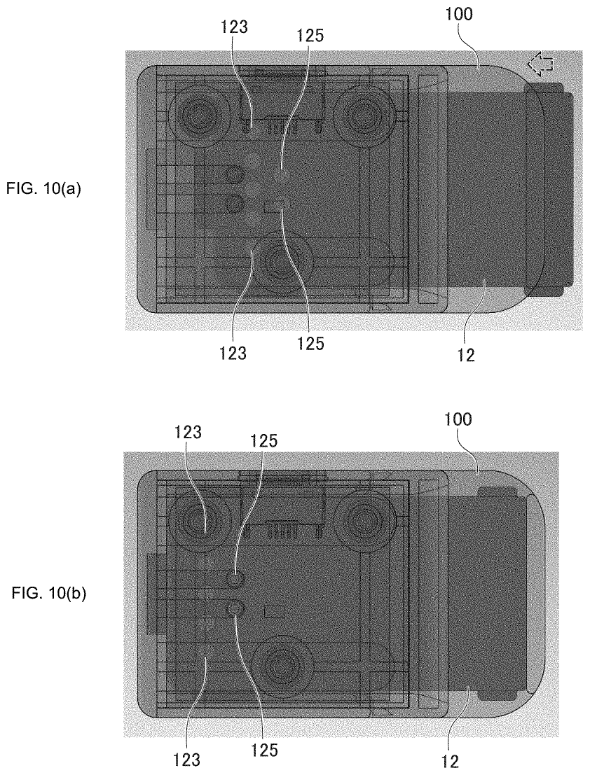

As illustrated in FIGS. 10(a) and 10(b), the plurality of (e.g., two) charging external terminals 125 are arranged in a row in or on the bottom surface of the main body part 12. Similarly, the plurality of (e.g., five) main-body-side external terminals 123 are arranged in a row in or on the bottom surface of the body part 12. The plurality of charging external terminals 125 and the plurality of main-body-side external terminals 123 are arranged such that their two rows are parallel to each other. Thus, a short-circuit between the charging external terminals 125 and the main-body-side external terminals 123 can be reliably prevented from occurring. Note that FIGS. 10(a)-(b) are diagrams illustrating the bottom surface (e.g., the external terminals 123 and 125) of the main body part 12. FIG. 10(a) illustrates the main body part 12 in the process of being set into the charger 100, and FIG. 10(b) illustrates the main body part 12 set in the charger 100.

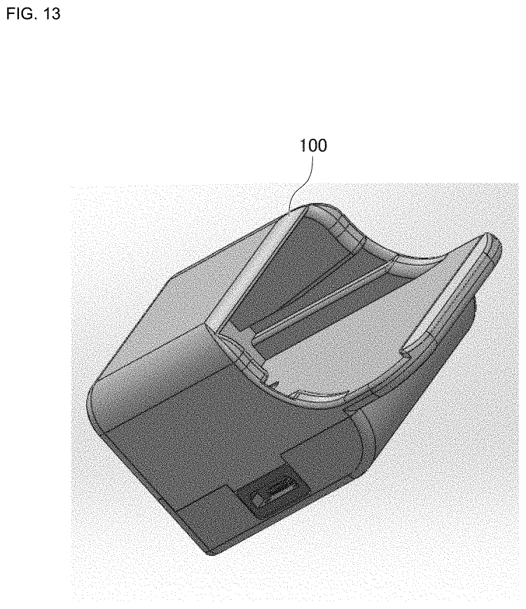

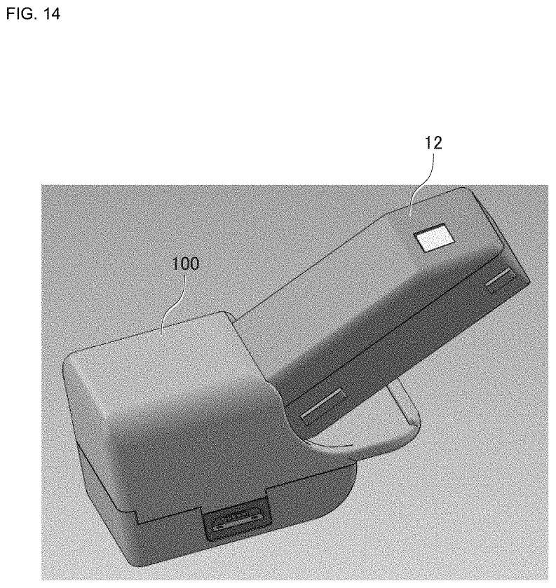

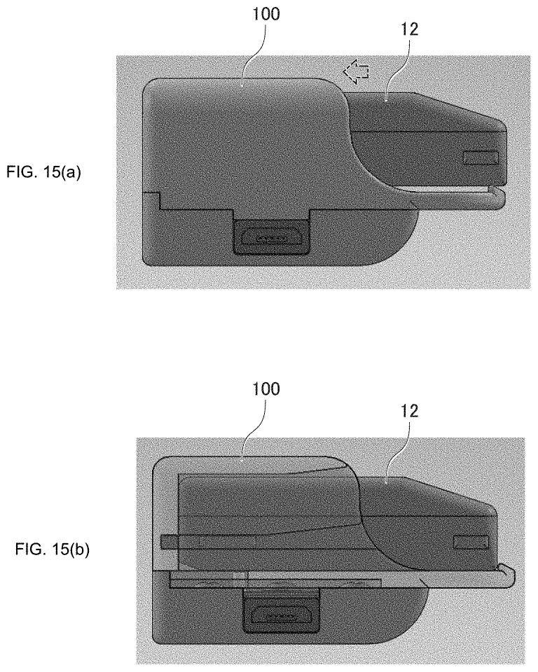

As illustrated in FIGS. 13, 14, and 15(a)-(b), the charger 100 has an opening through which the main body part 12, which is included in the biological-information acquisition device 1, can be inserted into the charger 100 and is configured so as to enable the main body part 12 to be (freely) attachable and detachable to and from the charger 100. The charger 100 charges the battery 122 through the charging external terminals 125 in a state where the main body part 12 is set in the charger 100. FIG. 13 is a perspective view illustrating an appearance of the charger 100 that charges the main body part 12 (e.g., the battery 122). FIG. 14 is a diagram illustrating a state in which the main body part 12 is in the process of being set into the charger 100. FIG. 15(a) is a side view illustrating the main body part 12 in the process of being set into the charger 100, and FIG. 15(b) is a view illustrating in a see-through manner the main body part 12 set in the charger 100.

As described in detail above, according to the present embodiment, the biometric sensor part 11, which is to be worn on a living body, is configured to be replaceable by being separated from the main body part 12. In other words, only the biometric sensor part 11 can be replaced, and the main body part 12 can be reused. This configuration makes it easier to replace the biometric sensor part 11 and to change the wearing form of the biometric sensor part 11 while suppressing an increase in the manufacturing costs. In addition, the main body part 12 is detachably attached to the biometric sensor part 11 so as not to come into contact with a living body when the biometric sensor part 11 is worn on the living body. Therefore, contamination of the main body part 12 can be prevented.

As a result, contamination of the main body part 12 can be prevented, and the biological-information acquisition device 1 can be flexibly adapted to various usage situations and individual differences while also decreasing the manufacturing costs.

In general, it is noted that although the exemplary embodiment has been described above, the present invention is not limited to the above-described embodiment, and various modifications can be made. For example, in the above-described embodiment, although a ring-type, an earphone-type, and an attachment-type have been mentioned as examples of the type of the biometric sensor part 11 (the living-body wearable part 112), the biometric sensor part 11 (living-body wearable part 112) of a different type may be employed.

In addition, in the above-described embodiment, although a body temperature sensor, a photoplethysmographic sensor, a pressure sensor, a blood glucose level sensor, an oxygen saturation sensor, a pulse wave sensor, and an electrocardiographic electrode have been mentioned as examples of the biometric sensors 111, different biometric sensors may be used instead of these sensors.

Furthermore, in the above-described exemplary embodiment, although biological information (e.g., a processing result) is transmitted in a wireless manner, it may be transmitted in a wired manner.

### REFERENCE SIGNS LIST

1, 1B biological-information acquisition device
11 biometric sensor part (biometric sensor unit)
111 biometric sensor
112 living-body wearable part
113 sensor-side external terminal
1131 biasing member
114 recess
115 accommodating portion
116 guide groove
117 curved portion
117a flexible substrate
118 flat portion
119 reinforcing plate
12 main body part (main body unit)
121 information processing section
1211 memory (storage unit)
1212 wireless communication module (wireless communication unit)
122 battery
123 main-body-side external terminal
1231 biasing member
124 hook portion
125 charging external terminal
100 charger

1. A biological-information acquisition device comprising:
a biometric sensor unit that includes a biometric sensor configured to detect biological information and a living-body wearable part that configures the biometric sensor to contact a living body; and
a main body that includes:
an information processor configured to process the biological information,
a battery configured to supply power to the information processing section,
a plurality of main-body-side external terminals that are electrically connected to the biometric sensor unit and that are arranged in a row in or on a bottom surface of the main body, and
a plurality of charging external terminals connected to a charger that charges the battery and that are arranged in a row in or on a bottom surface of the main body and parallel to the plurality of main-body-side external terminals,
wherein the biometric sensor unit is configured to be separated from the main body for replacement,
wherein the main body is detachably attached to the biometric sensor unit so that the main body does not contact the living body when the biometric sensor unit is worn on the living body.

2. The biological-information acquisition device according to claim 1,
wherein the biometric sensor unit is disposed in such a manner as to be exposed in a first direction in which a living body wearing the biometric sensor unit is located when viewed from the main body, and
wherein the main body is disposed in such a manner as not to be exposed in the first direction.

3. The biological-information acquisition device according to claim 2, wherein the biometric sensor unit includes a protruding portion exposed and protruding in the first direction.

4. The biological-information acquisition device according to claim 2, wherein the biometric sensor unit comprises a measuring surface that is exposed in the first direction and that is perpendicular to the first direction.

5. The biological-information acquisition device according to claim 2, wherein the main body is disposed in such a manner as to be exposed in a second direction that is opposite to the first direction.

6. The biological-information acquisition device according to claim 1, wherein the information processor includes:
a storage unit configured to store identification information, and
a wireless communication unit configured to transmit a processing result.

7. The biological-information acquisition device according to claim 1, wherein the biometric sensor unit includes a sensor-side external terminal that is electrically connected to the main body.

8. The biological-information acquisition device according to claim 7, wherein the sensor-side external terminal and the plurality of main-body-side external terminals are arranged in or on surfaces that face each other in a direction perpendicular to directions in which the main body is attached and detached to and from the biometric sensor unit.

9. The biological-information acquisition device according to claim 7,
wherein the sensor-side external terminal is disposed in or on a top surface of the biometric sensor unit,
wherein the plurality of main-body-side external terminals are disposed in or on a bottom surface of the main body, and
wherein the battery is disposed above the plurality of main-body-side external terminals.

10. The biological-information acquisition device according to claim 7, wherein at least one of the sensor-side external terminal and the plurality of main-body-side external terminals includes a biasing member configured to bias the respective terminal in a direction in which a distance between the sensor-side external terminal and the plurality of main-body-side external terminals is reduced.

11. The biological-information acquisition device according to claim 1, wherein the main body is engaged with the biometric sensor unit at three points.

12. The biological-information acquisition device according to claim 11,
wherein the main body includes three hooks on an outer surface of the main body in a protruding manner, and
wherein the biometric sensor unit is elastic and has three recesses into which the hooks of the main body are fitted.

13. The biological-information acquisition device according to claim 12,
wherein the biometric sensor unit has a recess in which the main body is accommodated, and
wherein a guide groove that guides a hook of the main body is formed in an inner surface of the recess, which is inclined with respect to a bottom surface thereof.

14. The biological-information acquisition device according to claim 1, wherein the living-body wearable part comprises a ring shape or an earphone shape.

15. The biological-information acquisition device according to claim 1,
wherein the living-body wearable part includes a curved portion that is wearable on a finger of the living body and a flat portion that connects respective ends of the curved portion, and
wherein the curved portion is provided with a flexible substrate on which the biometric sensor is mounted.

16. The biological-information acquisition device according to claim 15,
wherein the curved portion comprises an elastic material,
wherein the flat portion includes a reinforcing plate that has a rigidity higher than a rigidity of the curved portion.

17. The biological-information acquisition device according to claim 15, wherein the flat portion has a dimension larger than a dimension of the curved portion in an axial direction of the curved portion.

18. The biological-information acquisition device according to claim 15, wherein the battery is disposed such that a longitudinal direction of the battery is parallel to an axial direction of the curved portion.

19. A biological-information acquisition device comprising:
a biometric sensor unit that includes biometric sensor means for detecting biological information and means for configuring the biometric sensor means to contact a living body;
a main body that includes:
information processor means for processing the biological information,
a battery for supplying power to the information processing section,
a plurality of main-body-side external terminals that are electrically connected to the biometric sensor unit and that are arranged in a row in or on a bottom surface of the main body, and
a plurality of charging external terminals connected to a charger that charges the battery and that are arranged in a row in or on a bottom surface of the main body and parallel to the plurality of main-body-side external terminals; and
means for separating the biometric sensor unit from the main body for replacement,
wherein the main body is detachably attached to the biometric sensor unit so that the main body does not contact the living body when the biometric sensor unit is worn on the living body.

20. The biological-information acquisition device according to claim 19,
wherein the main body is engaged with the biometric sensor unit at three points,
wherein the main body includes three hooks on an outer surface of the main body in a protruding manner, and
wherein the biometric sensor unit is elastic and has three recesses into which the hooks of the main body are fitted.
